(19) United States
(12) Patent Application Publication
(10) Pub. No.: US 2005/0065365 A1
(43) Pub. Date: Mar. 24, 2005

(54) Process for making esters from butadiene

(75) Inventors: Darren John Cook, North Humberside (GB); Benjamin Patrick Gracey, Hull (GB)

Correspondence Address: Wallace L. Oliver, 22nd Floor, 120 South Riverside Plaza, Chicago, IL, 60606-3945, US

(73) Assignee: The Univ. of Southern Miss. Research Foundation, Hattiesburg, MS (US)

(21) Appl. No.: 10/952,326

(22) Filed: Sep. 28, 2004

Related U.S. Application Data
(63) Continuation of application No. PCT/GB03/01346, filed on Mar. 23, 2003.

(30) Foreign Application Priority Data
Apr. 2, 2002 (GB) 0207518

(51) Int. Cl. C07C 67/39

(52) U.S. Cl. 560/241

(57) ABSTRACT

A process for making a butyl ester from butadiene by reacting butadiene or a hydrocarbon fraction containing butadiene with a saturated aliphatic monocarboxylic acid, wherein the catalyst is rhenium(VII) oxide or an organic sulphonic acid containing at least 2 sulphonic acid groups per molecule wherein the ratio of the number of carbon atoms to the number of sulphonic acid groups in the organic sulphonic acid is in the range 1:1 to 1:0.15. Preferred catalysts are organic disulphonic acids for example ethane-1,2-disulphonic acid. The process can be used for making unsaturated esters, or, by hydrogenation of the product, for making saturated esters such as for example butyl acetate. Catalyst can be purified and recycled to the reactor.

### CROSS-REFERENCE TO RELATED APPLICATIONS

This application is a continuation of International Application No. PCT/GB03/01346, filed Mar. 23, 2003, incorporated by reference herein.

### BACKGROUND OF THE INVENTION

The present invention relates to a process for making esters by reacting butadiene with a carboxylic acid in the presence of a Brønsted or Lewis acid catalyst to form the unsaturated ester.

It is known that esters such as n-butyl acetate can be produced by a number of routes. For instance, the hydroformylation of propylene in the presence of acetic acid is a method which yields a mixture of n-butyl acetate and iso-butyl acetate. An alternative method is to react ethylene with vinyl acetate in the presence of an acid catalyst, followed by the hydrogenation of the resultant unsaturated ester. A further method is the reaction of ethylene with ethanol in the presence of a base catalyst to form butanol and the reaction thereof with acetic acid to form butyl acetate. The acid catalyzed addition of butadiene to acetic acid using ion-exchange resin catalysts having bulky counterions to improve the reaction selectivity to two isomeric C₄ butenyl acetates is disclosed in several patents viz., U.S. Pat. No. 4,450,288 (alkyl pyridinium), U.S. Pat. No. 4,450,287 (quaternary ammonium), U.S. Pat. No. 4,450,289 (quaternary phosphonium). Butadiene is a relatively inexpensive by-product of the refining process and is a potential feedstock for making butyl esters. It is commercially available either as a purified chemical or as a constituent of a hydrocarbon cut, for example, as a constituent of a mixed C₄ stream obtained from naphtha stream cracking. Typically such streams contain species such as butane, 1-butene, 2-butene, isobutane and isobutene in addition to butadiene. It is advantageous that a process using butadiene can use such streams. However, butadiene is also in equilibrium with 4-vinyl cyclohexene, a Diels Alder dimer of butadiene. This dimer can be thermally cracked back to butadiene:

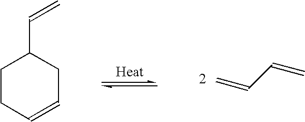

Thus, any process involving the use of butadiene as feedstock needs to take this reversible reaction into consideration.

EP-A-84133 describes a process for the production of unsaturated alcohols and/or esters of unsaturated alcohols. This document describes the reaction between conjugated dienes and water or aqueous carboxylic acids. The resulting product, is a complex mixture of unsaturated isomeric alcohols and esters.

U.S. Pat. No. 4,405,808 discloses a process for preparing esters of acetic acid by reacting acetic acid with an aliphatic lower olefin in the vapor phase in the presence of steam on a catalyst selected from the group consisting of an aromatic disulphonic acid and esters thereof. There is no disclosure of using a diene instead of an aliphatic lower olefin. The specific Examples in U.S. Pat. No. 4,405,808 relate only to the production of ethyl acetate and isopropyl acetate.

Our own WO 00/26175, incorporated by reference herein, discloses a process for making a butyl ester from butadiene which comprises, as a first step, the reaction of butadiene or a hydrocarbon fraction containing butadiene with a saturated aliphatic monocarboxylic acid, to form a mixture of n-butenyl and secondary butenyl esters.

The above process is said to be suitable for catalysis by either heterogeneous or homogeneous catalysts. Suitable homogeneous catalysts are said to include monosulphonic acids, triflic (trifluoromethanesulphonic) acid and its salts (triflates). Suitable organic sulphonic acids disclosed are methane sulphonic acid, p-toluene sulphonic acid and sulphonated calixarenes.

We have discovered that the above process can be performed with an improved combination of selectivity and yield by using certain specific catalysts.

### SUMMARY OF THE INVENTION

A butyl ester is made from butadiene by reacting butadiene or a hydrocarbon fraction containing butadiene with a saturated aliphatic monocarboxylic acid in a process wherein the catalyst comprises rhenium(VII) oxide or an organic sulphonic acid containing at least 2 sulphonic acid groups per molecule with ratio of the number of carbon atoms to the number of sulphonic acid groups in the range 1:1 to 1:0.15.

### BRIEF DESCRIPTION OF THE DRAWINGS

FIG. 1 is a process flowsheet illustrating the present invention.

FIG. 1A is a process flowsheet showing a scheme for purifying and/or concentrating the catalyst employed in the present invention.

FIG. 2 is a graph illustrating evolution of C₄ acetates from acetic acid/1,3-butadiene systems catalyzed by EDSA, NDSA, and ESA.

FIG. 3 is a graph illustrating evolution of C₄ acetates per acid group from acetic acid/1,3-butadiene systems catalyzed by EDSA, NDSA, and ESA.

FIG. 4 is a graph illustrating evolution of C₄ acetates from acetic acid/1,3-butadiene systems catalyzed by EDSA and MSA.

FIG. 5 is a graph illustrating evolution of C₄ acetates per acid group from acetic acid/1,3-butadiene systems catalyzed by EDSA and MSA.

### DESCRIPTION OF THE INVENTION

The present invention provides a process for making a butyl ester from butadiene, which comprises reacting butadiene or a hydrocarbon fraction containing butadiene with a saturated aliphatic monocarboxylic acid, wherein the catalyst comprises rhenium(VII) oxide or an organic sulphonic acid containing at least 2 sulphonic acid groups per molecule wherein the ratio of the number of carbon atoms to the number of sulphonic acid groups in the organic sulphonic acid is in the range 1:1 to 1:0.15.

In the sulphonic acid catalyst employed in the process of the present invention the ratio of the number of carbon atoms to sulphonic acid groups is preferably in the range 1:1 to 1:0.2, more preferably in the range 1:1 to 1:0.5 and most preferably in the range 1:1 to 1:0.7. The sulphonic acid preferably contains 2 to 30 carbon atoms, more preferably 2 to 10 carbon atoms and most preferably 2 to 8 carbon atoms.

The concentration of the defined sulphonic acid or rhenium oxide catalyst employed in the liquid phase of the reaction mixture can be maintained constant throughout the reaction, or can be varied or can be allowed to vary within a broad concentration range whilst still achieving desirable results.

The reaction can be carried out, for example, under batch conditions using a single aliquot of the defined sulphonic acid catalyst or rhenium oxide catalyst dissolved in some or all of the carboxylic acid, with butadiene gas being gradually pumped into the reaction. Under these conditions, the concentration of catalyst generally decreases due to the dilution effect as more and more butadiene enters the liquid phase with the formation of liquid ester. Alternatively, the reaction can be carried out by continuously or intermittently feeding in butadiene and or catalyst and/or carboxylic to maintain the concentrations of catalyst and reactants at the desired level. The catalyst can be fed in as solid or as a liquid. The catalyst fed to the reactor can be dissolved in solvent or in one of the reactants if desired, e.g. the catalyst can be dissolved in additional carboxylic acid if desired.

Preferably the catalyst concentration is maintained to contain 0.2 to 10 weight percent, preferably at least 0.5 to 7 wt %, most preferably 1 to 5 wt % of the sulphonic acid catalyst or the rhenium oxide catalyst based on the weight of the total reaction mixture. The sulphonic acid catalysts or rhenium oxide catalyst of the present invention are preferably soluble in the reaction mixture.

It is preferred that the reaction mixture forms a single liquid phase, but the reaction mixture can comprise two or more phases if desired.

Examples of suitable sulphonic acid catalysts are 1,2-ethane disulphonic acid, benzene-1,2-disulphonic acid, benzene-1,3-disulphonic acid, benzene-1,4-disulphonic acid, naphthalene-1,5-disulphonic acid, naphthalene-2,6-disulphonic acid, naphthalene-2,7-disulphonic acid, 4-chlorobenzene-1,3-disulphonic acid, 4-fluorobenzene-1,3-disulphonic acid, 4-bromobenzene-1,3-disulphonic acid, 4,6-dichlorobenzene-1,3-disulphonic acid, 2,5-dichlorobenzene-1,3-disulphonic acid, 2,4,6-trichlorobenzene-1,3-disulphonic acid, 3-chloronaphthalene-2,6-disulphonic acid, benzene trisulphonic acid, and naphthalene trisulphonic acid.

The defined sulphonic acid catalyst employed in the present invention contains at least two sulphonic acid groups per molecule. The defined sulphonic acid catalyst can comprise a single sulphonic acid compound or a plurality of different sulphonic acid compounds (each in accordance with the defined sulphonic acid) provided that the overall average carbon to sulphonic acid molar ratio for the defined sulphonic acid catalyst is in the range 1:1 to 1:0.15.

If desired, the defined sulphonic acid catalyst of the present invention can be used in admixture with one or more other catalysts known to be effective in catalyzing the addition reaction. For example, the defined sulphonic acid can be used in admixture with a monosulphonic acid catalyst. Preferably the defined sulphonic acid catalyst of the present invention forms at least 5 wt %, more preferably at least 10 wt %, most preferably at least 50 wt % of the total catalyst content. The use of catalysts containing from 80 to 100 wt % of the defined sulphonic acid catalyst is particularly preferred.

The butadiene employed in the invention may be employed in the form of a substantially pure butadiene. Alternatively, a hydrocarbon mixture comprising butadiene may be employed, for example, industrial hydrocarbon gas streams comprising butadiene. In one embodiment a raw (e.g. crude or depleted) C₄ stream comprising, inter alia, butadiene, isobutene, 1-butene, 2-butene and butane is employed. Such a stream may comprise up to 60% butadiene.

The saturated aliphatic monocarboxylic acid employed in the present invention is preferably a C₁ to C₃₀, more preferably a C₂ to C₁₅, most preferably a C₂ to C₆ acid. Acetic acid is particularly preferred.

The reaction is preferably carried out in the presence of water. For example, the liquid phase can contain 0.01 to 10 wt %, more preferably 0.05 to 5 wt % water based on the total liquid phase. Generally it has been found that low levels of water, e.g. in the range 0.1 to 5.0 wt % based on the total reaction mixture, are highly beneficial to the desired reaction. Thus it has been found that, at levels above 5% w/w the catalyst activity tends to be significantly reduced whereas at levels below 0.05% w/w, unacceptable selectivity loss can occur, although the activity is high. Consequently, the water level in the reaction zone suitably is in the range from 0.05 to 5% w/w on the carboxylic acid, preferably from 0.05 to 1% w/w. Also at high levels of water, hydrolysis of the product esters can become significant and the resultant product mixture containing, for example, allylic alcohols, can lead to additional expense on product work up.

The reaction is suitably carried out in the liquid or mixed liquid/gas phase in the presence of a solvent. The reaction is preferably carried out under conditions such that the reaction between the butadiene and the carboxylic acid occurs in the liquid phase. If a solvent is employed, it is not essential that both reactants dissolve completely in the solvent. However, it is an advantage if the solvent chosen is such that it is suitably capable of dissolving both the reactants. Specific examples of such solvents include hydrocarbons such as decane and toluene and oxygenated solvents such as glymes, ethers and esters, e.g. n-butyl acetate or excess carboxylic acid reactant and recycled higher esters such as C₈ acetates and recycled sec-butenyl acetate. The use of excess carboxylic acid as a reactant can be advantageous when the process of the present invention is used as part of a process for extracting butadiene from an impure stream, as it facilitates reaction at high conversion of butadiene, or in process terms, high efficiency of removal of butadiene. In current commercial practice, the removal or recovery of butadiene from refinery streams requires a separate processing stage.

In the process of the present invention it is also advantageous to use polymerization inhibitors such as alkylated phenols (e.g. BHT butylated hydroxytoluene, also called 2,6-di-tert-butyl-p-cresol). Other members of this series include the Irganox® series of materials from Ciba Gigy, Lowinox® series of materials from Great Lakes Chemical Corporation, Tropanol® series from ICl and t-butylcatechol, nitric oxide, nitroxides and derivatives (e.g. di-t-butylnitroxide, and n,n-dimethyl-4-nitrosoaniline,), stable radicals (e.g. 2,2,6,6,-tetramethyl-piperidine-1-oxyl, 2,2,6,6,-tetramethyl-4-hydroxypiperidine-1-oxyl and 2,2,6,6,-tetramethylpyrrolidine-1-oxyl).

The relative mole ratios of butadiene to the carboxylic acid reactant in the addition reaction is suitably in the range from 5:1 to 1:50, preferably in the range from 1:1 to 1:10.

The reaction is suitably carried out at a temperature in the range from 20 to 140° C., preferably from 20 to 130° C., more preferably 30 to 120° C., and most preferably 40 to 90° C. The reaction pressure is preferably the autogeneous reaction pressure which is determined by factors such as the reaction temperature, presence of absence of solvent, excess of reactants and impurities present in the butadiene stream. An additional pressure may be applied to the system if single fluid phase is preferred e.g. no butadiene gas phase in addition to the liquid phase (which may optionally contain a solvent).

The feed-stream to the reaction is preferably treated to remove corrosion metals and or basic impurities, for example ammonia or organic bases, therefrom. Such corrosion metals or bases can, for example, react with and inactivate the sulphonic acid catalyst.

Apparatus for carrying out the reaction will be well known to those skilled in the art. For example, the reaction may suitably be carried out in a plug flow reactor, a slurry reactor or a continuous stirred tank reactor. In the case that a plug flow reactor is employed, it is preferred that the unused butadiene is flashed off and recycled to the reactor via a vapor/liquid separator. In the case of a plug flow reactor, the butadiene can be present partially as a separate gas phase as well as being dissolved and this would permit operation, for example, in a trickle bed device or a bubble bed device. The butadiene feed can be added for example at a plurality of places in the reactor (e.g. at intervals along the length of a plug flow reactor). In the case of a bubble bed device, the butadiene can, if desired, be added counter-current to the carboxylic acid feed. A typical LHSV (liquid hourly space velocity=volume of liquid feed/catalyst bed volume) for the carboxylic acid is 0.1 to 20 more preferably 0.5 to 5. In the case of a continuous stirred tank reactor, a plurality of reactors in series can be employed if desired, and a continuous bleed of any deactivated catalyst can be taken. In this case it is economically advantageous to run with catalyst in a various stages of deactivation to improve the utilization of catalyst. This can result, for example, in the total loading of catalyst (activated+deactivated) reaching high levels, for example, 50% w/w or more of the reaction charge. Fresh catalysts can be added, continuously or intermittently if desired, to maintain the desired level of active catalyst in the reactor(s).

Preferably, the butadiene may be added gradually to the saturated aliphatic monocarboxylic acid, for example, by multiple injection at constant pressure in a batch reactor. By adding the butadiene gradually in this manner, side reactions leading to, for example, the polymerization of the butadiene can be minimized.

The addition reaction of the invention may optionally be followed by separation of the isomeric butenyl esters, i.e. the n-butenyl ester and secondary butenyl ester, as disclosed in our earlier application WO 00/26175. The sec-butenyl ester can be recovered and recycled to the initial addition reaction between butadiene and the carboxylic acid. It has been found that the sec-butenyl ester under reaction conditions interconverts with butadiene, free carboxylic acid, and the crotyl ester. The conversion of the sec-butenyl ester to free carboxylic acid and butadiene can be achieved, for example, by treatment in the vapor phase with an acidic support such as silica-aluminas. The use of such a separate pre-treatment prior to the return to the carboxylic acid and butadiene to the addition reactor can have a beneficial effect on productivity and selectivity.

The use of defined sulphonic acid catalysts of the present invention instead of monosulphonic acid catalysts for the addition reaction of acetic acid to 1,3-butadiene offers significant advantages in terms of activity. The resulting enhanced protonation of the 1,3-butadiene substrate offers a more favoured route to crotyl and sec-butenyl acetates for the sulphonic acid catalysts of the present invention as shown, for example, in the Examples below. Although the increased activity towards the addition reaction is tempered by a loss of selectivity in respect to the reagents, overall productivity is significantly greater for the sulphonic acid catalyst systems of the present invention.

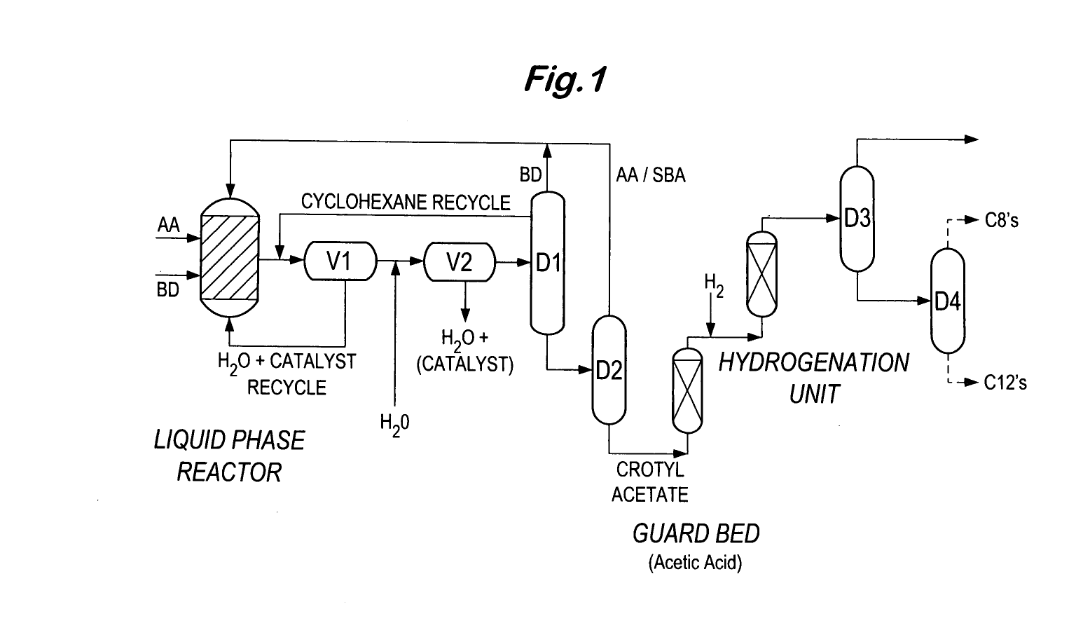

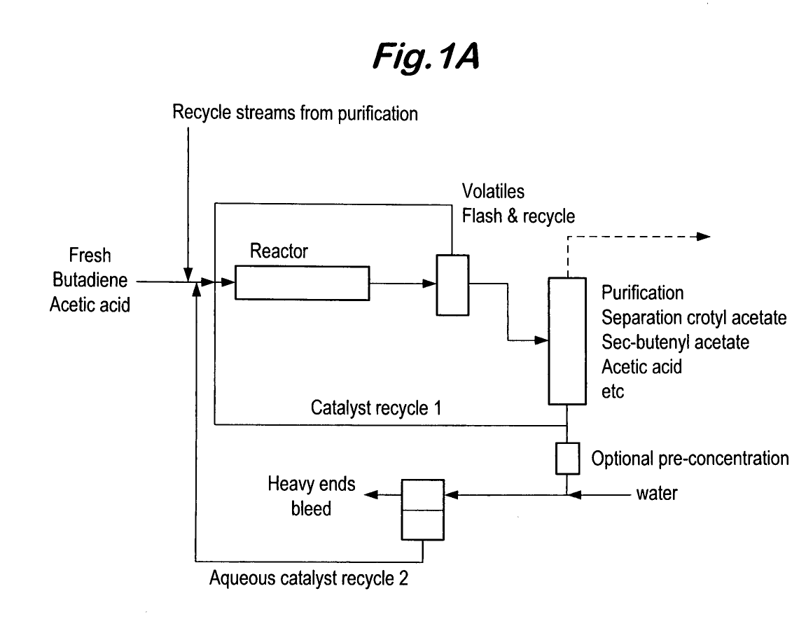

A theoretical process flowsheet is illustrated in FIG. 1. The liquid phase reactor is fed with butadiene and acetic acid in the presence of water and catalyst. A stream is taken and mixed with cyclohexane before separation in a decanter (V1). The aqueous phase is recycled to the reactor, whilst the cyclohexane containing aqueous phase is diluted with more water and passed into a second decanter (V2) where any residual catalyst is removed with the aqueous phase (to protect the preceding column from fouling). Column D1 flashes the butadiene over the heads for recycle into the reactor. A side draw is taken to pass the cyclohexane back into the stream from the reactor, whilst the stream from the column base (containing water, acetic acid, crotyl acetate, sec-butenyl acetate, and reaction byproducts) is fed to a second column (D2). This column will be operated to remove a crude crotyl acetate stream from the column base whilst water/acetic acid/sec-butenyl acetate distill over the heads and are recycled with the butadiene back to the reactor. The crude crotyl acetate stream is fed through a guard bed (to remove trace amounts of acid) and then hydrogenated. The desired butyl acetate stream can then be isolated from the residual heavy boilers by a final distillation. Distillation column D3 is provided for purifying the butyl acetate produced by hydrogenation of the crotyl acetate. Distillation column D4 is provided, if desired, for purifying C8 acetate ester by-products which can have value in their own right, or as alcohols obtained from the saponification of such esters.

FIG. 1A is a theoretical process flowsheet showing a scheme for purifying and/or concentrating the catalyst employed in the present invention. The addition reaction of the present invention tends to gradually produce small quantities of involatile by-products that, after long periods of operation, reduce the effective activity of the catalyst by dilution effect. In a preferred aspect of the present invention, the catalyst is continuously or intermittently purified by removal of such by-products. The purification process comprises diluting a portion of the reaction mixture with water, extracting organic materials from the resulting mixture by mixing with a water-immiscible solvent, optionally concentrating the resulting aqueous catalyst solution, and returning the catalyst solution to the reactor.

Thus FIG. 1A illustrates a scheme, applicable for example, to recycling the sulphonic acid catalyst to the reaction in the preparation of butenyl acetate from butadiene and acetic acid. The reaction is conducted in a vessel, which can be for example a bubble, tube, or stirred tank reactor. The exiting product stream consisting of unconverted reactants, reaction by-products and reaction products still contains active catalyst and, if left in contact with the reaction products for extended periods of time during the product work up, could catalyze the reverse reaction leading to liberation of butadiene, reactants and loss of product. Minimizing the contact time spent with the desirable reaction products with the catalyst under distillation conditions can reduce the degree of the reverse reaction. This can be facilitated by careful design of the distillation equipment in this example the reactant outlet mixture is first separated from the volatile components in a flash tank this generates a recycle stream of volatile components such as butadiene and vinyl cyclohexene, and then subjected to more extensive purification. The first stage of this purification could be a falling film, short path or wiped film evaporator. This resultant heavy ends stream contains as a high boiling point component the catalyst. It has been discovered that many of the high boiling reaction by-products are in dynamic equilibrium with the targeted reaction products and starting materials so it is adventitious to recycle many of these materials together with the catalyst. To one skilled in the art of process design, undesired impurities will build up in any recycle loop and this often necessitates a bleed off these materials. In the case of the catalyst recycle stream this could lead to a undesirable loss of expensive catalyst it has been discovered that liquid extraction of this residue stream allows recovery of the catalyst in an aqueous phase from the by-product oil phase. This separation can be facilitated by addition of a predominately water insoluble agent such as a hydrocarbon e.g. cyclohexane.

The invention is now illustrated with reference to the following Examples wherein Example 1 shows the reaction of the present in the production of butenyl acetate and Example 2 shows experimental details of a scheme for the extraction and purification of the catalyst.

### EXAMPLE 1

### Reaction of Butadiene with Acetic Acid

These experiments were undertaken in glassware adapted for batch reactions on a 100-gram total reactant scale, to run at around atmospheric pressure. Several experiments were run concurrently using this method to maximize reproducibility. 1,2-ethanedisulphonic acid and 1,5-naphthalenedisulphonic acid were compared with ethanesulphonic acid, a monosulphonic acid. Rhenium(VII) oxide was also tested. Reagents were used as supplied from Aldrich.

Typical reaction charge:-

| | |
|---|---|
| Acetic acid (containing BHT at ca. 500 ppm) | 97.9 g |
| Water | 0.1 g |
| Catalyst | 2.0 g |

The water content of the acetic acid feed was determined using a Karl-Fischer titration method (analysis repeated to a values within 0.05% w/w), and the level adjusted to that required. The reaction vessel was heated through a water jacket to 60° C. and loaded with acetic acid (pre-purged with nitrogen), water, inhibitor (BHT-2,6-di-tert-butyl-4-methylphenol or butylated hydroxytoluene) and catalyst in the required quantities. A magnetic stirrer bar was charged and the contents left to equilibrate with the bath temperature. 1,3-Butadiene was charged to the reaction vessel through a side arm from a liquid feed lecture bottle directly into the reaction liquor, and vented through a second side arm through a gas manifold designed to allow a slight overpressure of butadiene. The vessel was purged with butadiene for 10 minutes at a constant (set) flow rate. The point of this addition was taken as t=0 and the stirred autoclave contents were sampled at regular intervals and analyzed by flame ionization detector (FID) Gas chromatography (GC). The identity of the GC peaks was established by the synthesis of model compounds and GC/MS. The GC was calibrated by means of the purchase and synthesis of pure compounds i.e. acetic acid, butenyl acetate, sec-butenyl acetate, and 4-vinyl cyclohexene. The higher boiling by-products from the reaction were assigned the same response factor determined for butenyl acetate and thereby roughly quantified. All these higher boiling point material peaks were combined together—designated “highers”—and the calculated % w/w used to calculate the reaction selectivity.

Table 1 shows that the total C₄ acetate (sec-butenyl and crotyl acetate) yields for the monosulphonic acid ESA (ethanesulphonic acid) catalysed experiments are less than half those for equivalent experiments with the disulphonic acid EDSA (1,2-ethane disulphonic acid), calculated on a weight for weight loading basis (Runs 1 to 4). The ratio of sulphonic acid groups based on 2 grams of catalyst for ESA to EDSA is 5:6, which demonstrates a considerable enhancement of activity per sulphonic acid group in the disulphonic acid system. A further enhancement of sulphonic acid group activity is seen for 1,5-naphthalene disulphonic acid (Run 5). The effect of increasing water content on the evolution of C4 acetates differs for the two systems. A slight increase in activity is observed for ESA in moving from 0.3 to 1.0% water (ca. 11%), whilst DSA shows a decrease in activity (14%). The idea of assisted acidity between the close proximity acid sites would explain the activity decrease for EDSA, and is exemplified further by Run 6. Runs 7 and 9 show similar results for the same catalysts at lower loading (a slight decrease is observed in both cases on moving from 2 to 1% w/w). Rhenium(VII) oxide (Run 8) under the same conditions yields about 80% of C₄ acetates in comparison to EDSA, significantly more than ESA.

TABLE 1
Yield of C₄ acetates for butadiene/acetic acid addition (60° C.)

| | Run | | Catalyst | Water | 2-butenyl | Crotyl | Total C₄ | |
|---|---|---|---|---|---|---|---|---|
| Run | time | | charge | content | acetate | acetate | acetates | Temp. |
| No. | (h) | Catalyst | (% w/w) | (% w/w) | (% w/w) | (% w/w) | (% w/w) | (° C.) |
| 1 | 70 | ESA¹ | 2 | 0.2 | 1.203 | 1.136 | 2.339 | 60 |
| 2 | 70 | ESA¹ | 2 | 1.0 | 1.349 | 1.252 | 2.601 | 60 |
| 3 | 72 | EDSA² | 2 | 0.3 | 3.598 | 3.957 | 7.555 | 60 |
| 4 | 72 | EDSA² | 2 | 1.0 | 3.147 | 3.338 | 6.485 | 60 |
| 5 | 72 | NDSA⁴ | 2 | 0.3 | 2.976 | 3.212 | 6.188 | 60 |
| 6 | 72 | EDSA² | 2 | 4.0 | 1.012 | 0.956 | 1.968 | 60 |
| 7 | 70 | ESA¹ | 1 | 0.2 | 0.949 | 0.836 | 1.785 | 60 |
| 8 | 70 | RO³ | 1 | 0.3 | 2.028 | 2.137 | 4.165 | 60 |
| 9 | 72 | EDSA² | 1 | 0.3 | 2.609 | 2.817 | 5.426 | 60 |

¹ESA = ethanesulphonic acid (comparative run);
²EDSA = 1,2-ethanedisulphonic acid;
³RO = rhenium(VII) oxide;
⁴NDSA = 1,5-naphthalenedisulphonic acid.

Table 2 compares the selectivities of the homogeneous catalysts and relates them to their respective activities by calculating productivities. The selectivity, both with respect to acetic acid and to butadiene, decreases for equivalent systems on moving from monosulphonic acid to disulphonic acid groups. This suggests the assisted acidity issue for disulphonic acids not only enhances the evolution of C₄ acetates but also enhances routes to side-products to a greater extent i.e. enhanced acidity favours the formation of butadiene oligomers and C₈ acetates more than C₄ acetates. Rhenium(VII) oxide also shows poorer selectivity than ESA. By relating the selectivity and the activity, a productivity value can be assigned to the catalysts for comparisons to be made of the overall catalytic performance. These calculations indicate that EDSA exhibits a much better overall performance in generating C₄ acetates than ESA for the given set of reaction conditions. NDSA and rhenium(VII) oxide display productivity close to EDSA.

TABLE 2
Selectivity and productivity calculations

| | | | | | C₄ mol | C₄ mol | |
|---|---|---|---|---|---|---|---|
| | | | | | percent | percent | |
| | | | | | selectivity | selectiviy | |
| | | Catalyst | Water | Total C₄ | based on | based on | |
| | | charge | content | acetates | butadiene | acetic | |
| Run No. | Catalyst^{a} | (% w/w) | (% w/w) | (% w/w) | (%)^{b} | acid (%)^{c} | Productivity^{d} |
| 1 | ESA | 2 | 0.2 | 2.339 | 84.3 | 96.9 | 1.97 |
| 2 | ESA | 2 | 1.0 | 2.601 | 90.1 | 97.4 | 2.34 |
| 3 | EDSA | 2 | 0.3 | 7.555 | 71.6 | 92.4 | 5.41 |
| 4 | EDSA | 2 | 1.0 | 6.485 | 77.3 | 94.4 | 5.01 |
| 5 | NDSA | 2 | 0.3 | 6.188 | 75.9 | 94.1 | 4.70 |
| 6 | EDSA | 2 | 4.0 | 1.968 | 81.6 | 97.2 | 1.61 |
| 7 | ESA | 1 | 0.2 | 1.785 | 91.0 | 97.7 | 1.62 |
| 8 | RO | 1 | 0.3 | 4.165 | 75.3 | 93.8 | 3.14 |
| 9 | EDSA | 1 | 0.3 | 5.426 | 63.1 | 89.7 | 3.42 |

^{a}ESA - ethanesulphonic acid (110.13); EDSA - 1,2-ethanedisulphonic acid (FW 190.20); RO - rhenium(VII) oxide (FW 484.40); NDSA - 1,5-napthalenedisulphonic acid tetrahydrate (FW 360.36)
^{b}C₄ selectivity (with respect to butadiene) = [C₄ acetates]/([C₄ acetates] + 2[C₈ acetates] + 2[dimers] + [oligomers + trimers])
^{c}C₄ selectivity (with respect to acetic acid) = [C₄ acetates]/([C C₄ acetates] + [C₈ acetates])
^{d}Productivity = (selectivity to butadiene/100) * total C₄ acetates

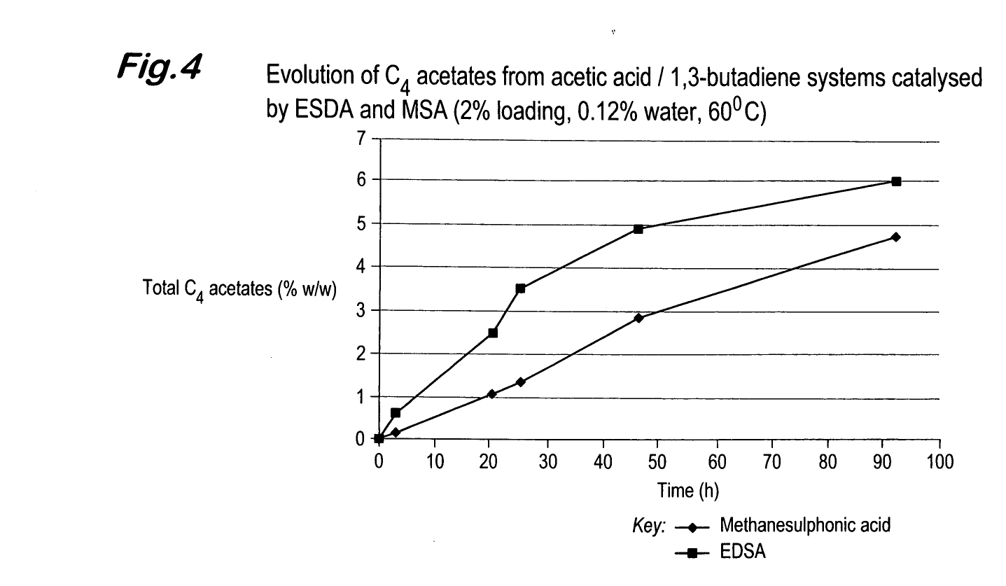

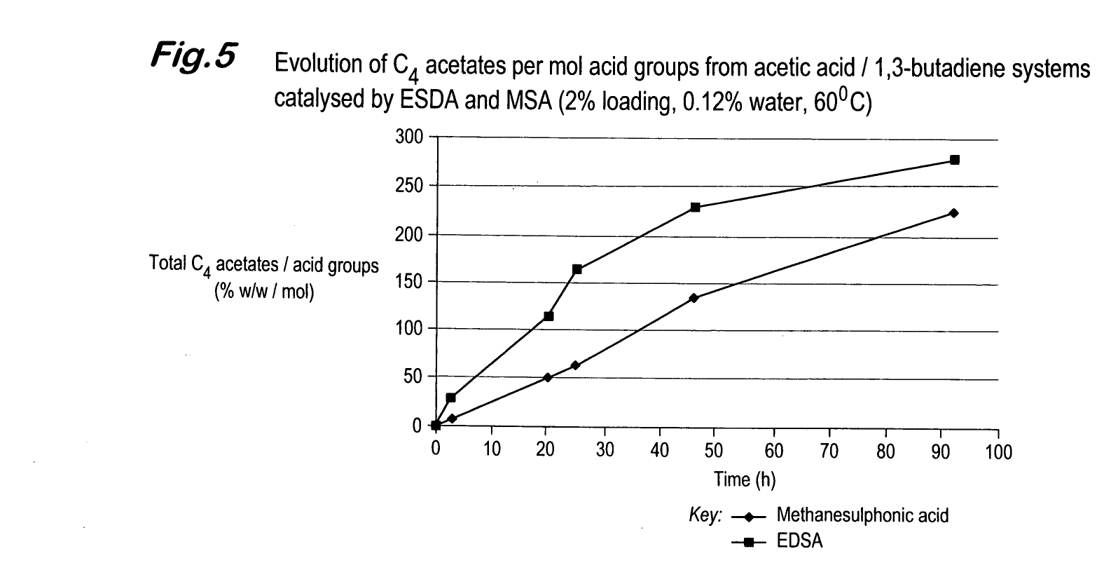

Similar experiments were undertaken to compare methanesulphonic acid (MSA) with EDSA (2% catalyst loading, 0.12% water, 60° C.), as illustrated in Tables 3 and 4 below and in FIGS. 4 and 5.

TABLE 3

| | Run | | Catalyst | Water | 2-Butenyl | Crotyl | Total C₄ | |
|---|---|---|---|---|---|---|---|---|
| Run | time | | charge | content | Acetate | Acetate | acetates | Temp. |
| No. | (h) | Catalyst^{a} | (% w/w) | (% w/w) | (% w/w) | (% w/w) | (% w/w) | (° C.) |
| 1 | 92 | EDSA | 2 | 0.12 | 2.718 | 3.297 | 6.015 | 60 |
| 2 | 92 | MSA | 2 | 0.12 | 2.197 | 2.578 | 4.775 | 60 |

^{a}EDSA -= 1,2-ethanedisulphonic acid; MSA = methanesulphonic acid

TABLE 4

| | | | | | C₄ mol % |
|---|---|---|---|---|---|
| | | | | | selectivity |
| | Catalyst | Water | Total C₄ | | based on |
| | charge | content | acetates | Reaction | acetic acid |
| Catalyst¹ | (% w/w) | (% w/w) | (% w/w) | time (h) | (%)² |
| EDSA | 2 | 0.12 | 6.015 | 92 | 99.8 |
| MSA | 2 | 0.12 | 4.775 | 92 | 99.9 |

¹EDSA = 1,2-ethanedisulphonic acid (FW 190.20); MSA = Methanesulphonic acid
²C₄ selectivity (with respect to acetic acid) = [C₄ acetates]/([C₄ acetates] + [C₈ acetates])

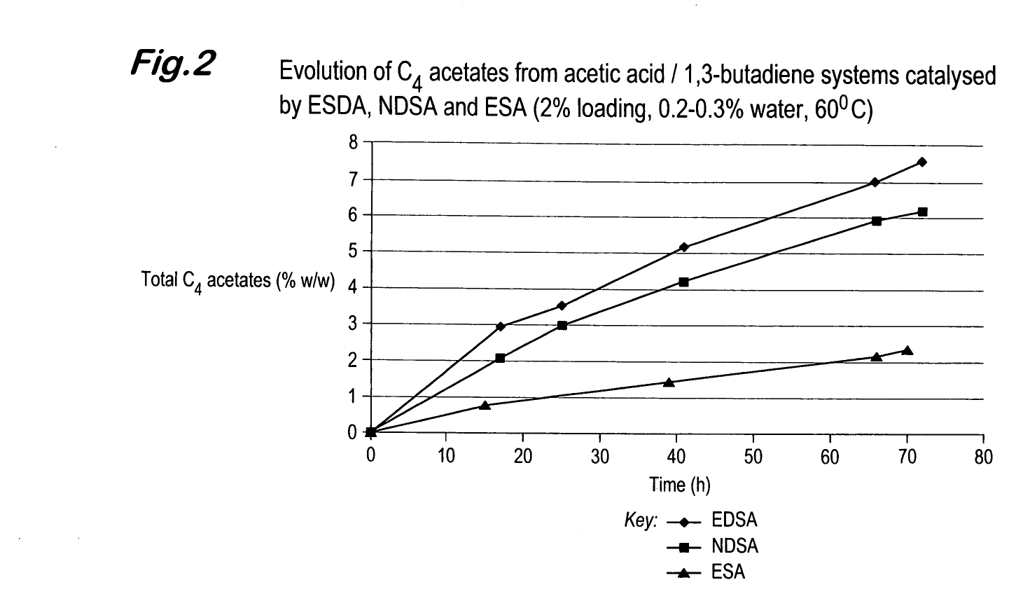

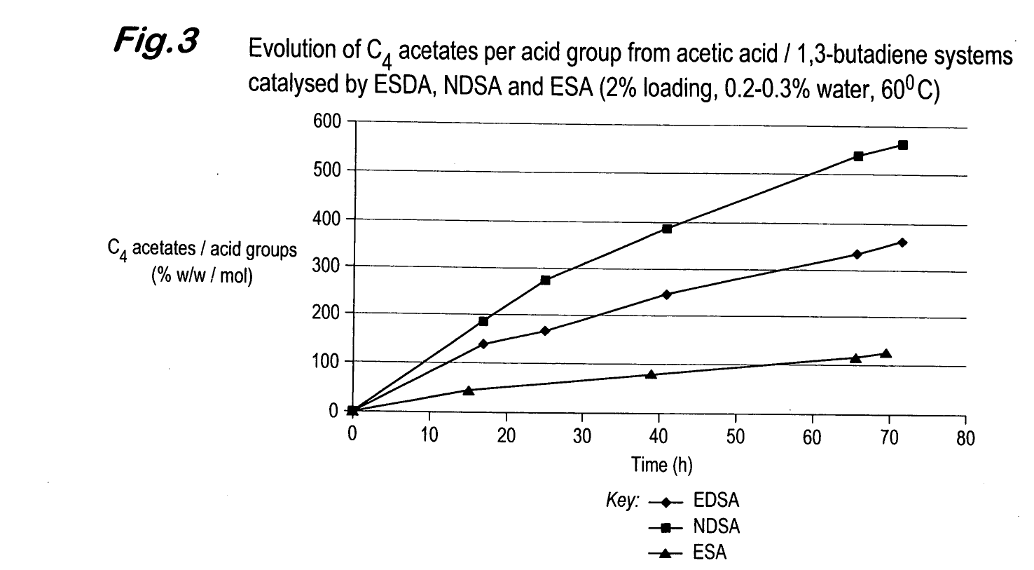

As for the previous systems, the initial evolution of C₄ acetates per acid group is much higher for disulphonic than monosulphonic acids, but in this system the C₄ acetate concentration approaches equilibrium more rapidly. Thus the final C₄ acetate levels in each system are much closer together than those in earlier runs (FIGS. 2 and 3). Clearly there is an advantage in using disulphonic acid catalysts over monosulphonic acid catalysts for the direct addition reaction, particularly at shorter reaction times (where equilibrium factors on reaction rate are less limiting).

### EXAMPLE 2

A series of seven consecutive batch reactions was conducted in which the catalyst was recovered and recycled by aqueous extraction.

The reagents were used as supplied by Aldrich Chemical Company without additional purification. Standard “Quickfit” laboratory glassware was used in these experiments. These experiments were conducted at atmospheric pressure unless mentioned otherwise.

For the initial run, a three-necked round bottomed flask was charged with 1,2-ethanedisulphonic acid (10.26 g, 2.07% w/w) and acetic acid (486.10 g, containing water at. 0.84% w/w by Karl-Fisher analysis) and mechanically stirred to aid dissolution (15 mins). The flask was connected to a butadiene inlet feed (from a lecture bottle) and glass bubbler vent outlet, and the apparatus placed upon a tared balance. A flow of butadiene was fed into the reaction until 30-40 g had been dissolved into the reaction media. The butadiene feed was then isolated and the flask then transferred to a water bath maintained at 60° C. by a “Haake” water bath and a mechanical stirrer attached through the central neck. The reaction was deemed to have started when the stirrer was switched on. After ca. 18 h a sample (ca. 3 g) was extracted from the reaction mixture via a syringe through a suba seal and used for GC analysis of the reaction components. The stirrer and water bath were turned off and the reaction allowed to cool.

Upon cooling, the catalyst was recovered by the following liquid-liquid extraction technique. The crude mixture was diluted with water (200 g) and extracted with cyclohexane (2×200 g). The aqueous acetic acid phase was charged to a Buchner flask and concentrated on a rotary evaporator (ca. 30 mBar, <50° C.) to an oil, when no more volatiles could be removed. The oil was dissolved in fresh acetic acid (490 g), the water level adjusted to ca. 1% when necessary, and the mixture transferred to the reaction flask for the subsequent run. A sample (ca. 3 g) was taken at this point to determine the initial composition of the charge (GC analysis). The flask was then charged with 30-40 g of butadiene (as before), and the run restarted. This process was repeated until the catalyst had been used 7 times.

### Runs 1-7

During these runs the sampling for analysis caused a loss of catalyst so the amount of catalyst was also measured to determine its specific activity.

GC analysis was undertaken on a Perkin-Elmer Autosystem chromatograph (100 m silica supported column) fitted with an autosampling facility. Decane (ca. 1%) was used as an internal standard to obtain compositions of C₄ acetate products and reaction by-products (primarily C₈ acetates and butadiene dimers/oligomers). Blank THF samples were run after every sample run to ensure elution of the heavier elements (e.g. butadiene trimers, oligomers) from the column. Results were quoted as percent weight for weight compositions of the total, gauged against the internal standard.

The cyclohexane extracts from each run were analyzed for sulphur to determine the extent of any catalyst leaching during the recycle process. Allowances were included in the calculations for catalyst lost during the sampling process. Results are shown in Tables 5-19.

TABLE 5
Mass balance data for Run 1

| Description | Weight (g) |
|---|---|
| Acetic acid charged (0.84% w/w) | 486.10 |
| EDSA charged | 10.26^{a} |
| Acetic acid + catalyst charged | 496.36 |
| Butadiene | 35.61 |
| Reaction mixture at the end of the run | 501.26 |
| Estimated butadiene uptake^{b} | 12.77 |
| Water added to mixture | 195.91 |
| Cyclohexane extract 1 | 198.52 |
| Cyclohexane recovered | 207.77 |
| Cyclohexane extract 2 | 136.73 |
| Cyclohexane recovered | 148.13 |
| Residue after removal of acetic acid/ | 13.11 |
| water | |
| Samples removed | 7.87^{c} |
| Catalyst lost through sampling | 0.16^{d} |

^{a}Catalyst concentration = 10.26/496.36 = 2.07% w/w
^{b}(Mixture at end + samples taken) − (Acetic acid + catalyst charged)
^{c}Samples removed = 100 × 7.87/496.36 = 1.58% of total
^{d}Catalyst lost = 1.58 × 10.26/100 = 0.16 g

TABLE 6
GC data for Run 1

| | | | | Sec- | |
|---|---|---|---|---|---|
| | | | Crotyl | Butenyl | Total C4 |
| | Time | | acetate (% | acetate (% | acetates |
| Sample | (h) | Weight (g) | w/w) | w/w) | (% w/w) |
| Run1-1 | 1 | 3.99 | 0.057 | 0.099 | 0.156 |
| Run1-2 | 18 | 3.88 | 1.108 | 1.049 | 2.157 |

TABLE 7
Run 2
Mass balance data for Run 1
Estimated catalyst concentration = 2.01%

| Description | Weight (g) |
|---|---|
| Acetic acid recharged (1.01% w/w) | 490.05 |
| Residue mass | 13.11 |
| Residue + acetic acid charge | 503.16 |
| Butadiene | 35.20 |
| Reaction mixture at the end of the run | 501.35 |
| Estimated butadiene uptake | 10.32 |
| Water added to mixture | 197.19 |
| Cyclohexane extract | 198.05 |
| Cyclohexane recovered | 203.26 |
| Cyclohexane extract 2 | 201.61 |
| Cyclohexane recovered | 215.63 |
| Residue after removal of acetic acid/ | 16.99 |
| water | |
| Samples removed | 12.13 |
| Catalyst lost through sampling | 0.24 |

TABLE 8
GC data for Run 2

| | | | Crotyl | Sec-Butenyl | Total C₄ |
|---|---|---|---|---|---|
| | Time | | acetate (% | acetate (% | acetates |
| Sample | (h) | Weight (g) | w/w) | w/w) | (% w/w) |
| Run2-1 | 0 | 3.52 | 0.062 | 0.06 | 0.122 |
| Run2-2 | 16 | 4.77 | 1.045 | 1.003 | 2.048 |
| Run2-3 | 18 | 3.84 | 1.3 | 1.21 | 2.51 |

TABLE 9
Run 3
Estimated catalyst concentration = 1.94%
Mass balance data for Run 3

| Description | Weight (g) |
|---|---|
| Acetic acid recharged (1.36% w/w) | 490.92 |
| Residue | 16.99 |
| Residue + acetic acid charge | 507.91 |
| Butadiene | 31.56 |
| Reaction mixture at the end of the run | 508.82 |
| Estimated butadiene uptake | 7.39 |
| Water added to mixture | 200.53 |
| Cyclohexane extract 1 | 198.13 |
| Cyclohexane recovered | 208.80 |
| Cyclohexane extract 2 | 198.42 |
| Cyclohexane recovered | 209.36 |
| Residue after removal of acetic acid/ | 12.33 |
| water | |
| Samples removed | 6.48 |
| Catalyst lost through sampling | 0.13 |

TABLE 10
GC data for Run 3

| | | | Crotyl | Sec-Butenyl | Total C₄ |
|---|---|---|---|---|---|
| | Time | | acetate | acetate | acetates |
| Sample | (h) | Weight (g) | (% w/w) | (% w/w) | (% w/w) |
| Run3-1 | 2 | 3.97 | 0.071 | 0.122 | 0.193 |
| Run3-2 | 18 | 2.51 | 0.87 | 0.851 | 1.721 |

TABLE 11
Run 4
Estimated catalyst concentration = 1.92%
Mass balance data for Run 4

| Description | Weight (g) |
|---|---|
| Acetic acid recharged (1.01% w/w) | 495.30 |
| Residue | 12.33 |
| Residue + acetic acid | 507.63 |
| Butadiene | 35.20 |
| Reaction mixture at the end of the run | 504.43 |
| Estimated butadiene uptake | 5.43 |
| Water added to mixture | 200.74 |
| Cyclohexane extract 1 | 201.60 |
| Cyclohexane recovered | 212.45 |
| Cyclohexane extract 2 | 200.48 |
| Cyclohexane recovered | 207.84 |
| Residue after removal of acetic acid/ | 32.99 |
| water | |
| Samples removed | 8.63 |
| Catalyst lost through sampling | 0.17 |

TABLE 12
GC data for Run 4

| | | | Crotyl | Sec-Butenyl | Total C4 |
|---|---|---|---|---|---|
| | Time | | acetate | acetate | acetates |
| Sample | (h) | Weight (g) | (% w/w) | (% w/w) | (% w/w) |
| Run4-1 | 0 | 4.04 | 0.053 | 0.049 | 0.102 |
| Run4-2 | 18 | 4.59 | 0.959 | 0.907 | 1.866 |

TABLE 13
Run 5
Estimated catalyst concentration = 1.82%
Mass balance data for Run 5

| Description | Weight (g) |
|---|---|
| Acetic acid recharged (1.78% w/w) | 492.62 |
| Residue | 32.99 |
| Residue + acetic acid | 525.61 |
| Butadiene | 37.43 |
| Reaction mixture at the end of the run | 527.44 |
| Estimated butadiene uptake | 9.27 |
| Water added to mixture | 199.97 |
| Cyclohexane extract 1 | 203.25 |
| Cyclohexane recovered | 214.94 |
| Cyclohexane extract 2 | 200.29 |
| Cyclohexane recovered | 213.53 |
| Residue after removal of acetic acid/ | 20.66 |
| water | |
| Samples removed | 7.44 |
| Catalyst lost through sampling | 0.14 |

TABLE 14
GC data for Run 5

| | | | Crotyl | Sec-Butenyl | Total C₄ |
|---|---|---|---|---|---|
| | Time | | acetate | acetate | acetates |
| Sample | (h) | Weight (g) | (% w/w) | (% w/w) | (% w/w) |
| Run5-1 | 0 | 3.44 | 0.053 | 0.049 | 0.102 |
| Run5-2 | 18 | 4.00 | 0.53 | 0.585 | 1.115 |

TABLE 15
Run 6
Estimated catalyst concentration = 1.84%
Mass balance data for Run 6

| Description | Weight (g) |
|---|---|
| Acetic acid recharged (1.61% w/w) | 490.46 |
| Residue | 21.34 |
| Residue + acetic acid | 511.80 |
| Butadiene | 30.35 |
| Reaction mixture at the end of the run | 516.60 |
| Estimated butadiene uptake | 13.77 |
| Water added to mixture | 200.05 |
| Cyclohexane extract 1 | 199.65 |
| Cyclohexane recovered | 209.64 |
| Cyclohexane extract 2 | 201.46 |
| Cyclohexane recovered | 212.33 |
| Residue after removal of acetic acid/ | 12.08 |
| water | |
| Samples removed | 8.97 |
| Catalyst lost through sampling | 0.17 |

TABLE 16
GC data for Run 6

| | | | Crotyl | Sec-Butenyl | Total C₄ |
|---|---|---|---|---|---|
| | Time | | acetate | acetate | acetates |
| Sample | (h) | Weight (g) | (% w/w) | (% w/w) | (% w/w) |
| Run6-1 | 0 | 4.57 | 0.000 | 0.000 | 0.000 |
| Run6-2 | 18 | 4.40 | 0.764 | 0.786 | 1.550 |

TABLE 17
Run 7
Estimated catalyst concentration = 1.84%
Mass balance data for Run 7

| Description | Weight (g) |
|---|---|
| Acetic acid recharged (0.98% w/w) | 490.70 |
| Residue | 11.92 |
| Residue + acetic acid | 502.62 |
| Butadiene | 32.33 |
| Reaction mixture at the end of the run | 504.37 |
| Estimated butadiene uptake | 10.47 |
| Water added to mixture | 200.22 |
| Cyclohexane extract 1 | 202.81 |
| Cyclohexane recovered | 245.47 |
| Cyclohexane extract 2 | 208.45 |
| Cyclohexane recovered | 214.91 |
| Residue after removal of acetic acid/ | 14.79 |
| water | |
| Samples removed | 8.72 |
| Catalyst lost through sampling | 0.16 |

TABLE 18
GC data for Run 7

| | | | Crotyl | Sec-Butenyl | Total C4 |
|---|---|---|---|---|---|
| | | | acetate (% | acetate (% | acetates (% |
| Sample | Time (h) | Weight (g) | w/w) | w/w) | w/w) |
| Run7-1 | 0 | 4.57 | 0.004 | 0.000 | 0.004 |
| Run7-2 | 18 | 4.15 | 0.872 | 0.819 | 1.681 |

TABLE 19
Results for sulphur content of the cyclohexane extracts from
successive regeneration experiments Runs 1-7

| | Weight of | Sulphur | Acid | | |
|---|---|---|---|---|---|
| | cyclohexane | content in | weight in | | Calculated |
| | layer as | organic layer | organic | | acid in |
| | discharged | (mg/kg) | layer (mg/ | Acid weight in | aqueous |
| Sample | (g) | [uncertainty] | kg) | organic layer (g) | layer (g) |
| Run3-1C1 | 207.77 | 0.6 [0.2] | 3.6 | 0.017 | 10.083 |
| Run3-1C2 | 148.13 | 0.4 [0.2] | 2.4 | 0.016 | 10.067 |
| Run3-2C1 | 203.26 | 0.6 [0.2] | 3.6 | 0.017 | 9.810 |
| Run3-2C2 | 215.63 | 0.7 [0.2] | 4.2 | 0.019 | 9.791 |
| Run3-3C1 | 208.80 | 0.6 [0.2] | 3.6 | 0.017 | 9.644 |
| Run3-3C2 | 209.36 | 0.6 [0.2] | 3.6 | 0.017 | 9.627 |
| Run3-4C1 | 212.45 | 0.3 [0.2] | 1.8 | 0.009 | 9.448 |
| Run3-4C2 | 207.84 | 0.3 [0.2] | 1.8 | 0.009 | 9.439 |
| Run3-5C1 | 214.94 | 0.5 [0.2] | 3.0 | 0.014 | 9.285 |
| Run3-5C2 | 213.53 | 0.5 [0.2] | 3.0 | 0.014 | 9.271 |
| Run3-6C1 | 209.64 | 0.3 [0.2] | 1.8 | 0.009 | 9.092 |
| Run3-6C2 | 212.33 | 0.5 [0.2] | 3.0 | 0.014 | 9.078 |
| Run3-7C1 | 245.47 | 0.5 [0.2] | 3.0 | 0.012 | 8.906 |
| Run3-7C2 | 214.91 | 0.4 [0.2] | 2.4 | 0.011 | 8.895 |
| Run3-7F1 | N/A¹ | 1700 [100] | 1699 | 10.092² [9.500-10.686] | N/A |
| Run3-7F2 | N/A¹ | 1600 [100] | 1599 | 9.500² [8.904-10.092] | N/A |
| Run3-7F3 | N/A¹ | 1600 [100] | 1599 | 9.500² [8.904-10.092] | N/A |

Mass of sulphur = 32 g/mol
Mass of 1,2-ethanedisulphonic acid = 190.2 g/mol
Scaling factor = 190.2/32.0 = 5.94
¹The residue (14.79 g) after volatiles were removed in vacuo was dissolved in acetone (1000.51 g) and analyzed for sulphur.
²Calculated acid mass in residue.

1-20. (cancelled)

21. A process for making a butyl ester from butadiene comprising reacting butadiene or a hydrocarbon mixture containing butadiene with a saturated aliphatic monocarboxylic acid, wherein the catalyst comprises a rhenium(VII) oxide or an organic sulphonic acid containing at least 2 sulphonic acid groups per molecule in which the ratio of the number of carbon atoms to the number of sulphonic acid groups in the organic sulphonic acid is in the range 1:1 to 1:0.15.

22. A process as claimed in claim 21 wherein the catalyst comprises the sulphonic acid and the ratio of the number of carbon atoms to sulphonic acid groups is in the range 1:1 to 1:0.2.

23. A process as claimed in claim 21 wherein the catalyst comprises the sulphonic acid and the ratio of the number of carbon atoms to sulphonic acid groups is in the range 1:1 to 1:0.7.

24. A process as claimed in claim 21 wherein the sulphonic acid catalyst contains 2 to 30 carbon atoms.

25. A process as claimed in claim 21 wherein the catalyst is soluble in the reaction mixture or in a component of the reaction mixture.

26. A process as claimed in claim 21 wherein the reaction mixture contains 0.2 to 10 weight percent of the catalyst based on the weight of the total reaction mixture.

27. A process as claimed in claim 24 wherein the sulphonic acid catalyst is selected from 1,2-ethane disulphonic acid, benzene-1,2-disulphonic acid, benzene-1,3-disulphonic acid, benzene-1,4-disulphonic acid, naphthalene-1,5-disulphonic acid, naphthalene-2,6-disulphonic acid, naphthalene-2,7-disulphonic acid, 4-chlorobenzene-1,3-disulphonic acid, 4-fluorobenzene-1,3-disulphonic acid, 4-bromobenzene-1,3-disulphonic acid, 4,6-dichlorobenzene-1,3-disulphonic acid, 2,5-dichlorobenzene-1,3-disulphonic acid, 2,4,6-trichlorobenzene-1,3-disulphonic acid, 3-chloronaphthalene-2,6-disulphonic acid, benzene trisulphonic acid, and naphthalene trisulphonic acid.

28. A process as claimed in claim 24 wherein the saturated aliphatic monocarboxylic acid employed in the present invention is a C₂ to C₆ acid.

29. A process as claimed in claim 21 wherein the reaction is carried out in the liquid or mixed liquid/gas phase in the presence of a solvent.

30. A process as claimed in claim 29 wherein the solvent is a hydrocarbon solvent.

31. A process as claimed in claim 21 wherein a polymerization inhibitor is included in the reaction.

32. A process as claimed in claim 31 wherein the polymerization inhibitor is an alkylated phenol.

33. A process is claimed in claim 21 wherein the relative mole ratio of butadiene to the carboxylic acid reactant in the addition reaction is in the range from 5:1 to 1:50.

34. A process is claimed in claim 33 wherein the relative mole ratio of butadiene to the carboxylic acid reactant in the addition reaction is in the range from 1:1 to 1:10.

35. A process as claimed in claim 21 wherein the reaction is carried out in the presence of excess carboxylic acid reactant as solvent.

36. A process as claimed in claim 21 wherein the reaction is carried out in a plug flow reactor.

37. A process as claimed in claim 21 wherein the reaction is carried out in a continuous stirred tank reactor.

38. A process as claimed in claim 21 wherein the catalyst is the sulphonic acid.

39. A process as claimed in claim 21 wherein the catalyst is the rhenium (VII) oxide.

40. A process has claimed claim 21 wherein the reaction temperature is in the range 30 to 120° C. and is carried out under autogenous pressure.
